(19) United States
(12) Patent Application Publication
(10) Pub. No.: US 2026/0232945 A1
(43) Pub. Date: Aug. 13, 2026

(54) DIFFUSER DEVICE

(71) Applicants: Larry Nouvel, Plano, TX (US); John Laurence Strauss, Mies (CH); Stephan Laurence Strauss, Ecublens (CH)

(72) Inventors: Larry Nouvel, Plano, TX (US); John Laurence Strauss, Mies (CH); Stephan Laurence Strauss, Ecublens (CH)

(21) Appl. No.: 19/153,522

(22) PCT Filed: Feb. 2, 2024
(86) PCT No.: PCT/US2024/014247

§ 371 Date: Aug. 4, 2025

Related U.S. Application Data
(63) Continuation of application No. 18/105,652, filed on Feb. 3, 2023.

(51) Int. Cl. A61M 16/14; A01K 15/00; A61K 9/00; C09K 3/30

(52) U.S. Cl. CPC A61M 16/14; A01K 15/00; A61K 9/0043; C09K 3/30

(57) ABSTRACT

The present disclosure provides for non-electrical diffuser devices for use in the treatment of pets or other animals, the device comprising a container for holding a formulation, an outlet, and a passive diffusing element mounted across the outlet for controlling discharge of the formulation from the container.

### CROSS-REFERENCE TO RELATED APPLICATIONS

This application is a 371 National Phase application of PCT/US2024/014247, filed Feb. 2, 2024, which claims priority to U.S. patent application Ser. No. 18/105,652, filed Feb. 3, 2023, the contents of which are hereby expressly incorporated by reference in their entirety and for all purposes.

### INTRODUCTION

This present disclosure relates generally to diffuser devices for use with calming/behavioral pheromones. In at least one embodiment, the present disclosure relates to a non-electrical diffuser device for use with calming/behavioral pheromones for treatment of pets or other animals.

### BACKGROUND

Pet behavioral/calming pheromones are available today in a variety of delivery systems, collars, snacks, toys, sprays and mainly heated plug-in devices for treating a large area such as a room. The reason for these applications, and especially for heated plug-in devices, is because the current pheromones are not highly volatile. A number of plug-in diffusers are currently available for evaporating a formulation into a space, such as a room, to bring about a calming effect on pets, for example. These diffusers typically provide a plug-in device for mounting at a standard electrical socket to power a heater within the device to evaporate the formulation for diffusion throughout the room over a prolonged period of time. Various stand-alone diffuser devices for mounting on furniture, such as a cabinet or table or on a mantlepiece or window ledge for example, are available and include a battery, or rechargeable battery or can be plugged in to a power supply to operate the built-in heater to evaporate and diffuse the formulation. A disadvantage of these devices is their use is limited to a specific space/room. Also, they require a power supply to diffuse the formulation.

The Applicant has discovered an improved diffuser device that does not require a power supply for the release of calming/behavioral pheromones.

### BRIEF SUMMARY

According to the present disclosure there is provided a diffuser device, comprising: a container for reception of a formulation, the container having (i) an outlet, and (ii) a passive diffusing element mounted across the outlet, the diffusing element being operable to control discharge of the formulation from the container.

In one embodiment of the present disclosure, the diffusing element comprises a membrane mounted across the outlet, the membrane being permeable to at least a portion of the formulation in vapor form to control discharge of said portion of the formulation.

In another embodiment, the diffusing element is a wick. In this case the container may conveniently comprise a glass or plastic bottle of any desired size, typically up to about 500 ml, the wick extending within the bottle from the outlet to a bottom of the bottle. The wick may conveniently comprise polyethylene or a polyethylene terephthalate material or any other suitable material.

Advantageously the formulation is sufficiently volatile to passively diffuse in ambient temperature (typically about 10° C. to about 40° C.) and does not require a heater for diffusion, thus saving on manufacturing cost and running cost, and affords greater flexibility in positioning/locating the diffuser device for use. The membrane provides for a controlled diffusion of the formulation over a prolonged period, typically up to about 30 days and possibly more, and can be controlled through size of the membrane surface and type of membrane or volume or type of formulation.

In another embodiment the pheromone is highly volatile at room temperature.

In another embodiment the vapor pressure of the pheromone is in the range of about 16.5 to about 25 mmHg at 25° C. and preferably is about 17.105 mmHg at 25° C.

In another embodiment the formulation comprises at least one pheromone. It is envisaged that the pheromone will comprise about 0.001 to about 5% by weight of the formulation.

In another embodiment the pheromone may be for relief of a medical condition such as stress, motion sickness, diet control and the like. That is, for example, the pheromone may produce a calming effect on pets.

In another embodiment the pheromone may be trans-2-methyl-2-butenal.

In another embodiment the formulation can include a fragrance and/or a color.

In another embodiment, the formulation can include a solvent.

In another embodiment, the formulation can be a micro-emulsion.

In another embodiment, the formulation can include paraffin wax.

In another embodiment the formulation can be provided in a liquid or gel form.

In another embodiment, the formulation can include a gelling agent.

In one embodiment of the present disclosure the container, tray or holder can be vacuum-formed from a multi-layer plastics sheet.

In another embodiment the container, tray or holder may have a first layer comprising PET (polyethylene terephthalate), a second layer comprising polyethylene and a third layer comprising copolymer EVOH (ethylene-vinyl alcohol copolymer). Other layered plastics films are also possible.

In another embodiment the membrane can comprise polyethylene, polypropylene or other material.

In another embodiment a peel-away sealing sheet can be mounted across an outer face of the membrane to seal the membrane before use.

In another embodiment the peel-away sealing sheet may comprise an aluminium foil and/or plastics material.

In another embodiment, a holder can be provided upon which the container is supported or held.

In another embodiment the holder could be a table-top stand to treat a room for example, or a compact unit such as a pendant which can be worn by a pet or other animal. Thus advantageously, a pet wearing the compact unit would always be in the presence of the area of treatment by the pheromone, not just when they are in a specific treated space or room. Alternatively, the diffuser device may be adapted for attachment to veterinary Elizabethan collars. In this case a base of the container may be at least partially coated with adhesive covered by a peel-away cover sheet.

The holder can be constructed of any suitable material, including metal, plastics, wood and cardboard.

In another embodiment, the container can be a plastic or glass container with a wick extending to the bottom of the container.

### BRIEF DESCRIPTION OF THE DRAWINGS

The present technology will be more clearly understood from the following description of certain embodiments thereof, given by way of example only, with reference to the accompanying drawings, wherein:

FIG. 1 is a schematic sectional elevational view of a diffuser device according to the present disclosure;

FIG. 2 is a detail elevational view of a diffuser membrane forming portion of the diffuser device; and

FIG. 3 is a perspective view of another diffuser device according to a second embodiment of the present disclosure;

FIG. 4 is a perspective view of a diffuser device according to a third embodiment of the present disclosure;

FIG. 5 is a schematic sectional elevational view of the diffuser device shown in FIG. 4;

FIG. 6 is a perspective view of a diffuser device according to a fourth embodiment the present disclosure;

FIG. 7 is an exploded perspective view of the diffuser device shown in FIG. 6; and

FIG. 8 is a perspective view of a packaging carton for the diffuser device of FIG. 6.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout this disclosure will now be presented. The term “about” means reasonably close to the particular value. For example, about does not require the exact measurement specified and can be reasonably close. As used herein, the word “about” can include the exact number. The terms “comprising,” “including” and “having” are used interchangeably in this disclosure. The terms “comprising,” “including” and “having” mean to include, but not necessarily be limited to the things so described. In this specification the terms “comprise, comprises, comprised and comprising” or any variation thereof and the terms “include, includes, included and including” or any variation thereof are considered to be totally interchangeable and they should all be afforded the widest possible interpretation and vice versa.

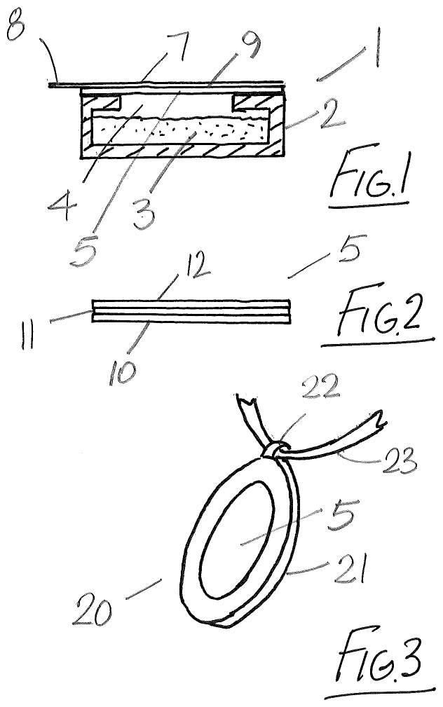

The disclosure now turns to FIG. 1 and FIG. 2, which illustrate a diffuser device 1 according to the present disclosure. The diffuser device 1 can include a container 2 for reception and storage of a volatile formulation 3 in liquid or gel form and further may contain a selected pheromone that is sufficiently volatile to passively diffuse at ambient temperature. An outlet 4 of the container 2 can be covered by a membrane 5 which is permeable to the volatile formulation 3 when in vapor form to diffuse the vapor through the membrane 5 in a controlled manner over an extended period of time, typically about 30 days or more. The formulation may, for example, be chosen to bring about a calming or behavioral effect on pets. Different formulations to achieve different effects on animals are possible. A peel-away aluminium foil sealing sheet 7 with pull tab 8 can cover an external face 9 of the membrane 5.

The pheromone may be selected to alleviate a particular medical condition such as stress, motion sickness, appetite control, or the like. For example, the pheromone may be selected to bring about a calming effect on pets. The pheromone may be highly volatile at room temperature having a vapor pressure in the range of about 16.5 to about 25 mmHg at 25° C. and preferably is about 17.105 mmHg at 25° C. The pheromone may comprise 0.001-5% by weight of the formulation. In certain embodiments, the pheromone may be trans-2-methyl-2-butenal.

FIG. 2 illustrates that the container 2 can be formed using a multi-layer plastics sheet. In certain embodiments, the container 2 can be vacuum-formed from a multi-layer plastics sheet, wherein the multi-layer plastics sheet may have an inner first layer 10 comprised of PET, an intermediate second layer 11 comprised of polyethylene, and an outer third layer 12 comprised of copolymer EVOH. The membrane 5 may be comprised of polypropylene material.

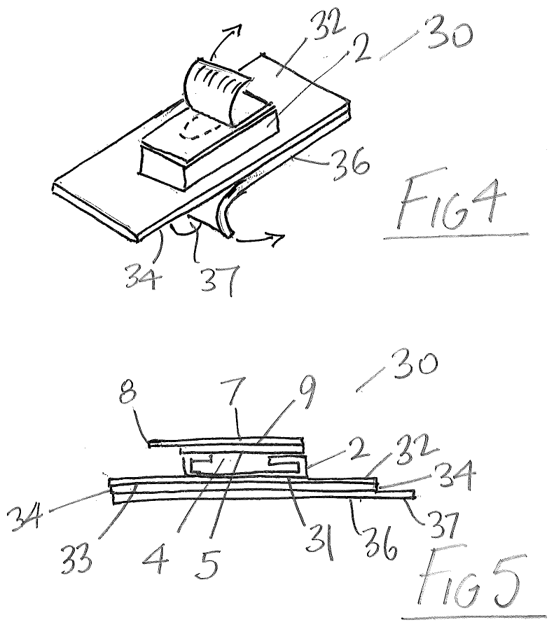

Referring now to FIG. 3 there is illustrated a diffuser device according to a second embodiment of the present disclosure, indicated generally by the reference numeral 20. Parts similar to those described previously are assigned the same reference numerals. In this case the container 2 can be mounted within a holder, which can be in the form of a pendant 21 having a loop 22 for reception of a tie 23, which can be a cord or chain for example, so that the pendant 21 can be worn about a pet's neck or attached to a pet's collar. Thus, the pet can constantly be in the presence of the active pheromone vapor.

Referring now to FIG. 4 and FIG. 5, there is illustrated a diffuser device according to a third embodiment of the present disclosure, indicated generally by the reference numeral 30. Parts similar to those described previously are assigned the same reference numerals. In this case, a bottom 31 of the container 2 can be attached to a holder formed by a mounting strip 32. An underside 33 of the mounting strip 32 can be coated with a layer of adhesive 34. A peel-away cover sheet 36 with outwardly projecting grip tab 37 can cover the adhesive layer 34 prior to use. Double-sided adhesive tape could alternatively be used.

After removing the cover sheet 36, the device 30 can be attached to a collar, such as an Elizabethan collar or other type of pet collar. The foil sealing sheet 7 can be removed to gradually diffuse the pheromone from the container 2 to maintain a calming atmosphere surrounding the pet wearing the collar. In an alternative arrangement the mounting strip 32 could be attached to a support surface such as a table, a wall or a window in a room or vehicle frequented by the pet.

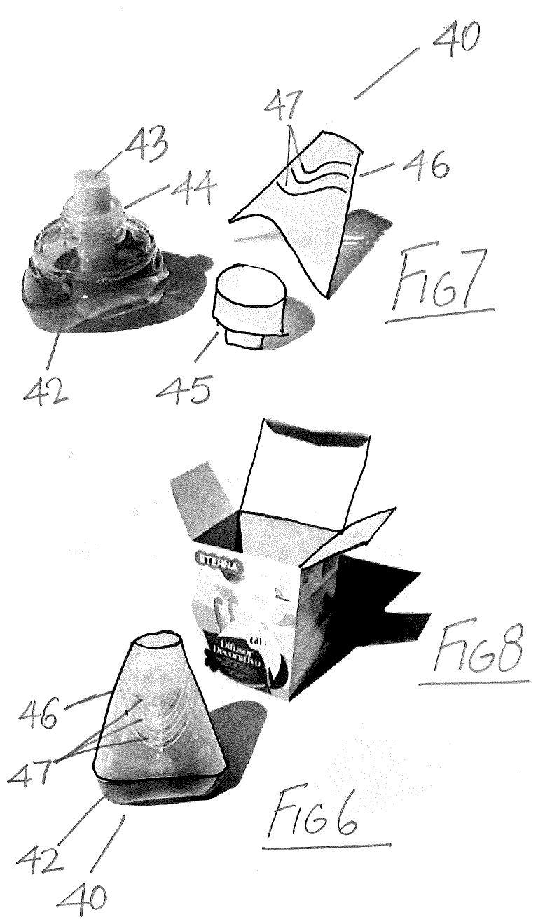

Referring now to FIG. 6 to FIG. 8, there is illustrated a diffuser device according to a fourth embodiment of the present disclosure, indicated generally by the reference numeral 40. Parts similar to those described previously are assigned the same reference numerals. In this case the container 2 can comprise a plastics or glass bottle 42 which contains the formulation. A wick 43 forming the diffusing element can be mounted in the neck 44 outlet of the bottle 42 and can extend within the bottle 42 to the bottom of the bottle 42. A cap 45 can engage with the neck 44 to seal the bottle 42 prior to use or when not in use. Upon removal of the cap 45 a decorative diffuser cowl 46 with vent openings 47 can engage and sit on the bottle 42 housing the wick 43 within the cowl 46. The formulation can be gradually dispensed from the bottle through the wick 43. Diffusion of the formulation can be stopped by replacing the cap 45 on the bottle 42.

The size of the container used can be chosen depending on the volume of formulation required, the desired length of treatment and the area to be treated.

The device according to the various embodiments of the present disclosure does not require a power supply for the release of calming/behavioral pheromones. The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

1. A diffuser device, comprising:
a container for reception of a formulation which is sufficiently volatile to passively diffuse in ambient temperature;
the container having an outlet; and
a passive diffusing element mounted across the outlet, the diffusing element operable to control discharge of the formulation from the container.

2. The diffuser device of claim 1, wherein the diffusing element comprises a membrane mounted across the outlet, the membrane being permeable to at least a portion of the formulation in vapor form to control discharge of said portion of the formulation.

3. The diffuser device of claim 1, wherein the diffusing element is a wick.

4. The diffuser device of claim 3, wherein the container is a bottle, the wick extending within the bottle from the outlet to a bottom of the bottle.

5. The diffuser device of claim 3, wherein the wick comprises polyethylene or a polyethylene terephthalate material.

6. The diffuser device of claim 1, wherein the formulation includes a pheromone which is highly volatile at room temperature.

7. The diffuser device of claim 6, wherein the vapor pressure of the pheromone is in the range of about 16.5 to about 25 mmHg at 25° C.

8. The diffuser device of claim 6, wherein the pheromone comprises about 0.001-to about 5% by weight of the formulation.

9. The diffuser device of claim 6, wherein the pheromone is for relief of a medical condition such as stress, motion sickness, diet control and the like.

10. The diffuser device of claim 9, wherein the pheromone provides a calming effect on pets.

11. The diffuser device of claim 6, wherein the pheromone is trans-2-methyl-2-butenal.

12. The diffuser device of claim 1, wherein the formulation includes a fragrance and/or a color.

13. The diffuser device of claim 1, wherein the formulation includes a solvent.

14. The diffuser device of claim 1, wherein the formulation includes paraffin wax.

15. The diffuser device of claim 1, wherein the formulation is a micro-emulsion.

16. The diffuser device of claim 1, wherein the formulation is provided in a liquid or gel form.

17. The diffuser device of claim 1, wherein the formulation includes a gelling agent.

18. The diffuser device of claim 1, wherein the container is vacuum-formed from a multi-layer plastics sheet.

19. The diffuser device of claim 18, wherein the container comprises a first layer comprising PET (polyethylene terephthalate), a second layer comprising polyethylene and a third layer comprising copolymer EVOH (ethylene-vinyl alcohol copolymer).

20. The diffuser device of claim 2, wherein the membrane comprises polyethylene or polypropylene.

21. The diffuser device of claim 20, wherein a peel-away sealing sheet is mounted across an outer face of the membrane to seal the membrane before use.

22. The diffuser device of claim 21, wherein the peel-away sealing sheet comprises an aluminium foil and/or plastics material.

23. The diffuser device of claim 1, wherein a holder is provided upon which the container is supported or held.

24. The diffuser device of claim 23, wherein the holder is a table-top stand.

25. The diffuser device of claim 23, wherein the holder is a compact unit such as a pendant which can be worn by a pet.

26. The diffuser device of claim 1, wherein the diffuser device is adapted for attachment to veterinary Elizabethan collars (E-collars), a base of the container being at least partially coated with adhesive covered by a peel-away cover sheet.
